(19) United States
(12) Patent Application Publication
(10) Pub. No.: US 2025/0231739 A1
(43) Pub. Date: Jul. 17, 2025

(54) APPARATUS AND METHOD FOR REFINING AND PREPROCESSING BIO-SIGNAL DATA

(71) Applicant: ELECTRONICS AND TELECOMMUNICATIONS RESEARCH INSTITUTE, Daejeon (KR)

(72) Inventors: Hyeon Jeong LEE, Daejeon (KR); Min Seong KIM, Daejeon (KR)

(73) Assignee: ELECTRONICS AND TELECOMMUNICATIONS RESEARCH INSTITUTE, Daejeon (KR)

(21) Appl. No.: 18/955,924

(22) Filed: Nov. 21, 2024

(30) Foreign Application Priority Data
Jan. 16, 2024 (KR) 10-2024-0006971

(51) Int. Cl. G06F 5/01; G16H 10/60

(52) U.S. Cl. CPC G06F 5/01; G16H 10/60

(57) ABSTRACT

The present invention relates to an apparatus and method for refining and preprocessing bio-signal data. The apparatus for refining and preprocessing bio-signal data includes a communication unit that receives bio-signal data or medical data, and a processor that generates segments with a certain length from the bio-signal data, refines the data by determining a data value, quality, and validity of each of the segments, converts and normalizes the refined data to pre-process the data, and generates a dataset for learning or evaluation based on the refined and preprocessed data, thereby effectively processing a large amount of data by standardizing and automating the process of refining and preprocessing the bio-signal data with various characteristics.

### CROSS-REFERENCE TO RELATED APPLICATION

This application claims priority to and the benefit of Korean Patent Application No. 10-2024-0006971, filed on Jan. 16, 2024, the disclosure of which is incorporated herein by reference in its entirety.

### BACKGROUND

### 1. Field of the Invention

The present invention relates to an apparatus and method for refining and preprocessing bio-signal data that refine and preprocess single or multiple bio-signal data according to the same standard.

### 2. Discussion of Related Art

As we enter an aging society, there is growing interest in early diagnosis and prevention of various diseases, including cardiovascular disease. In addition, the supply of wearable devices is increasing along with the demand for smart healthcare that measures and monitors an individual's health status anytime, anywhere in daily life.

Bio-signal data obtained through the wearable device often shows different characteristics depending on a person. In addition, due to the nature of the bio-signal data being vulnerable to noise caused by the external environment, movement, or the like, a process of refining and preprocessing data should be performed before analyzing and utilizing the bio-signal data.

For example, when the bio-signal data such as photoplethysmography (PPG) mixed with arrhythmia, noise, or the like is used without appropriately refining and preprocessing the bio-signal, the accuracy of blood pressure estimation can decrease. In addition, the development of systems for estimating, determining, and predicting a user's condition by analyzing such bio-signal data using machine learning and artificial neural network algorithms is actively underway in various fields.

In order to utilize the machine learning and artificial neural network algorithms in the analysis of the bio-signal data, a large amount of data having the same standard and form is required.

However, there is a problem in that the bio-signal data measured and collected with various sensors or devices in various environments are mostly different from each other and may not be directly used as input for the machine learning or artificial neural network. In addition, there is a problem in that the bio-signal data should individually undergo the process of refining and preprocessing data to secure usable data, which consumes a long time and high cost.

Therefore, an apparatus for refining and preprocessing bio-signal data is required to secure and process a large amount of high-quality bio-signal data.

Accordingly, a method of refining and driving data, such as Korean Patent No. 10-2097741, entitled “System for Refining Medical Image Data of Training Artificial Intelligence and Driving Method Thereof,” has been proposed.

However, there is a problem in that the method, which is a method of refining medical images, cannot be applied to refining of bio-signal data.

### SUMMARY OF THE INVENTION

The present invention is directed to providing an apparatus for refining and preprocessing bio-signal data capable of standardizing and automating a process of refining and preprocessing the bio-signal data collected through wearable sensors or devices to generate the bio-signal data having the same standard and make the generated bio-signal data into a database, and a method therefor.

According to an aspect of the present invention, there is provided an apparatus for refining and preprocessing bio-signal data, which includes: a communication unit that receives bio-signal data or medical data; and a processor that generates segments with a certain length from the bio-signal data, refines the data by determining a data value, quality, and validity of each of the segments, converts and normalizes the refined data to pre-process the data, and generates a dataset for learning or evaluation based on the refined and preprocessed data.

The processor may classify the bio-signal data based on at least one of units of records, units of the segments, and units of patients of the medical data and refine the data.

The processor may identify the length of the data in the units of the records to determine suitability of use of the data for training data.

The processor may identify the length of the data in the units of the patients, and determine validity to determine suitability of use of the data for training data.

The processor may identify cases in which the data overlaps, is missed, and includes an outlier at a certain length or a certain frequency of the segment to determine the quality depending on use of the training data.

When the data of the segment is repeated or the signal has a certain pattern, the processor may interpolate the segment using specific values of an interval within the segment through an interpolation method.

The processor may interpolate the segment using at least one of an average value, a maximum value, a minimum value, and a median value based on neighboring values in an interval within the segment.

The processor may preprocess the bio-signal data by performing frequency conversion to standardize a sampling rate of the bio-signal data and normalization to constantly adjust a signal size.

The processor may acquire features of time, frequency, and nonlinear domains of the signal for the bio-signal data during a preprocessing process.

The processor may feed the features back to a process of refining data to perform secondary refining.

The processor may extract metadata from data generated during a refining and preprocessing process of the bio-signal data.

The processor may sample refined and preprocessed bio-signal data to process the data, convert the processed data into training data to generate a dataset, and manage the dataset using the metadata as an index.

According to another aspect of the present invention, there is provided a method of refining and preprocessing bio-signal data, which includes: acquiring, by a communication unit, bio-signal data or medical data; generating, by a processor, segments with a certain length from the bio-signal data; determining, by the processor, a data value, quality, and validity of each of the segments to refining the data; converting and normalizing, by the processor, the refined data to preprocess the data; and generating and storing, by the processor, a dataset for learning or evaluation based on the refined and preprocessed data.

The refining of the data may include refining the bio-signal data in units of records, and the processor may identify a length of the data in the units of the records to determine suitability of use of the data for training data.

The refining of the data may include refining the bio-signal data in units of the segments, and the refining of the bio-signal in the units of the segments may include identifying cases in which the data overlaps, is missed, and includes an outlier at a certain length or a certain frequency of the segment to determine the quality depending on the use of training data.

When the data of the segment is repeated or the signal has a certain pattern, the refining of the bio-signal in the units of the segments may include interpolating the segment using specific values of an interval within the segment through an interpolation method.

The refining of the bio-signal in the units of the segments may further include interpolating the segment using at least one of an average value, a maximum value, a minimum value, and a median value based on neighboring values in the interval within the segment.

The refining of the data may include refining the bio-signal data in the units of the patients of the medical data, and the processor may identify a length of the data in the units of the patients, and determine validity to determine suitability of use of the data for training data.

In the preprocessing of the data, the processor may preprocess the bio-signal data by performing frequency conversion to standardize a sampling rate of the bio-signal data and normalization to constantly adjust a signal size.

The preprocessing of the data may include: acquiring, by processor, features of time, frequency, and nonlinear domains of the signal for the bio-signal data; and feeding the features back to a process of refining data to perform secondary refining.

### BRIEF DESCRIPTION OF THE DRAWINGS

FIG. 1 is a block diagram schematically illustrating a control configuration of an apparatus for refining and preprocessing bio-signal data according to an embodiment of the present invention.

FIG. 2 is a flowchart illustrating a data flow of the apparatus for refining and preprocessing bio-signal data according to an embodiment of the present invention.

FIG. 3 is a flowchart illustrating a process of refining data of the apparatus for refining and preprocessing bio-signal data according to an embodiment of the present invention.

FIG. 4 is a flowchart illustrating a process of refining and preprocessing data of the apparatus for refining and preprocessing bio-signal data according to an embodiment of the present invention.

FIG. 5 is a flowchart illustrating a method of refining and preprocessing bio-signal data according to an embodiment of the present invention.

### DETAILED DESCRIPTION OF EXEMPLARY EMBODIMENTS

Hereinafter, embodiments of an apparatus and method for refining and preprocessing bio-signal data according to the present invention will be described. In this process, thicknesses of lines, sizes of components, and the like, illustrated in the accompanying drawings may be exaggerated for clearness of explanation and convenience. In addition, terms to be described below are defined in consideration of functions in the present invention and may be construed in different ways by the intention of users or practice. Therefore, these terms should be defined on the basis of the contents throughout the present specification.

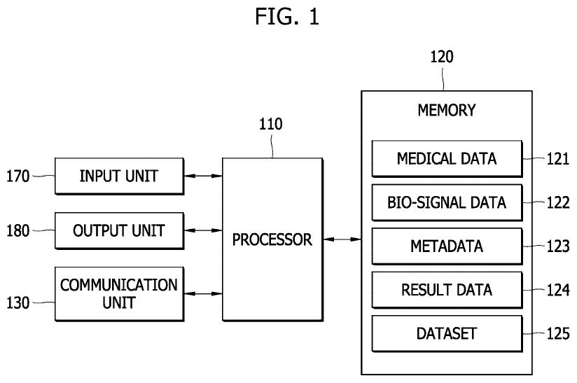

FIG. 1 is a block diagram schematically illustrating a control configuration of an apparatus for refining and preprocessing bio-signal data according to an embodiment of the present invention.

Referring to FIG. 1, an apparatus 100 for refining and preprocessing bio-signal data according to an embodiment of the present invention may include an input unit 170, an output unit 180, a communication unit 130, a memory 120, and a processor 110.

The memory 120 may store medical data 121, bio-signal data 122, metadata 123, result data 124, and a dataset 125.

The medical data 121 is data including medical information collected through the communication unit 130 and the input unit 170. The medical data 121 may include a sex, an age, presence of cardiovascular disease, a disease diagnosis code, etc., of a subject. In addition, the medical data 121 includes treatment, diagnosis, and prescription information generated by medical institutions, and may include statistics and lifestyle data collected from medical devices, wearable devices, IoT devices, sensors, mobile apps, etc.

The bio-signal data 122 is data acquired through the wearable device, the sensor, etc. For example, the bio-signal data 122 may include electrocardiogram (ECG), photoplethysmography (PPG), and electromyogram (EMG). The PPG data may be measured from various parts of a body, and may be used in a wide variety of areas, such as estimating blood pressure, calculating heart rate and oxygen saturation, measuring stress, and determining arrhythmia, since cardiovascular conditions are reflected in pulse waves.

The metadata 123 is data extracted during the preprocessing of the bio-signal data 122. The result data 124 is the refined and preprocessed bio-signal data. The dataset 125 is data that is classified and grouped based on the metadata 123 extracted during the preprocessing and refining of the data.

The memory 120 may store data related to at least one of a data preprocessing algorithm, a data refining algorithm, a learning algorithm, and a data analysis algorithm.

The memory 120 may include storage means such as non-volatile memory such as a random access memory (RAM), a read-only memory (ROM), and an electrically erased programmable ROM (EEPROM), a flash memory, a hard disk drive (HDD), a solid state drive (SSD), and a software-defined storage (SDS).

In some cases, the memory 120 may be configured as a database.

The input unit 170 may receive the medical data 121 and the bio-signal data 122. The input unit 170 may receive a user's command for preprocessing and refining of data and transmit the received user's command to the processor 110. The input unit 170 may include at least one input means among a switch, a button, and a touch pad.

The output unit 180 may output a data input/output status, and progress status and results during the preprocessing and refining of the data. The output unit 180 may include at least one of a speaker, an operating lamp, and a display.

The communication unit 130 may communicate with an external terminal (not illustrated), a server (not illustrated), and a database (not illustrated) through a communication network.

The communication unit 130 may receive the medical data 121 and the bio-signal data 122 from an external server or a terminal. In addition, the communication unit 130 may be connected to the wearable device, the sensor, and a medical device sensor through a communication terminal and receive the measured data or pre-stored bio-signal data.

The communication unit 130 may transmit the metadata 123, the result data 124, and the dataset 125 to the database.

The communication unit 130 may transmit and receive data in a wired or wireless communication manner. The communication unit 130 may perform communication through at least one of short-range communication such as Ethernet, WiFi, and Bluetooth, mobile communication, and serial communication.

The processor 110 may include at least one microprocessor and may operate according to an algorithm stored in the memory 120.

The processor 110 may preprocess and refine the bio-signal data acquired through the input unit 170 or the communication unit 130 and make the preprocessed and refined bio-signal data into a database. The processor 110 may preprocess and refine the bio-signal data and standardize the preprocessed and refined bio-signal data according to the same standard.

The processor 110 may preprocess and refine the bio-signal data 122 and the medical data 121 and extract the metadata 123 in the process.

The processor 110 may generate dataset based on the metadata 123, make the generated dataset into a database, and store the generated dataset.

The processor 110 receives single or multiple multi-channel bio-signal data acquired through the communication unit 130 or receives the bio-signal data through the input unit 170 and processes the bio-signal data.

The processor 110 may match the bio-signal data 122 and the related medical data 121 to process data.

The processor 110 divides the bio-signal data 122 into pieces of data with a certain length to generate segments.

The processor 110 may evaluate the quality of the acquired bio-signal data 122 and determine suitability of use of the bio-signal data 122 for machine learning and artificial neural network learning. The processor 110 may evaluate quality in units of segments.

The processor 110 may determine that data is not suitable for learning for at least one of when the data overlaps, when the data is missed, and when the data includes outliers at a certain length (interval) or a certain frequency (number of times) or more.

When the segment of the bio-signal data 122 is not suitable for data analysis, model learning, development, etc., the processor 110 may convert the segment into a usable form.

In this case, the processor 110 may interpolate a signal when a segment in which data overlaps or a segment in which a certain signal is repeated among the segments that are not suitable for learning.

In addition, when a segment is not suitable for learning, the processor 110 may classify the segment as a segment not to be used. The processor 110 may use the segment, which is not suitable for learning, as a negative sample.

The processor 110 may perform conversion, normalization, filtering, and data analysis based on the medical data 121 for the refined bio-signal data 122 in the time and frequency domains to preprocess the data.

In addition, the processor 110 may classify the bio-signal data 122 as a specific group based on the medical data 121 and preprocess the bio-signal data 122. The processor 110 may classify the bio-signal data and then refine and preprocess the data differently for each group.

The processor 110 may extract information on intermediate data and final result data produced during the refining and preprocessing as the metadata 123.

The processor 110 may store the refined and preprocessed bio-signal data as the result data 124 and extract the metadata 123 from the result data 124. The processor 110 may manage the dataset 125 for the bio-signal data 122 based on metadata 123 according to certain criteria.

The processor 110 may perform sampling on the preprocessed bio-signal data 122 and metadata 123, and convert the sampled data into the data form that can be provided to the machine learning or artificial neural network to generate the dataset 125.

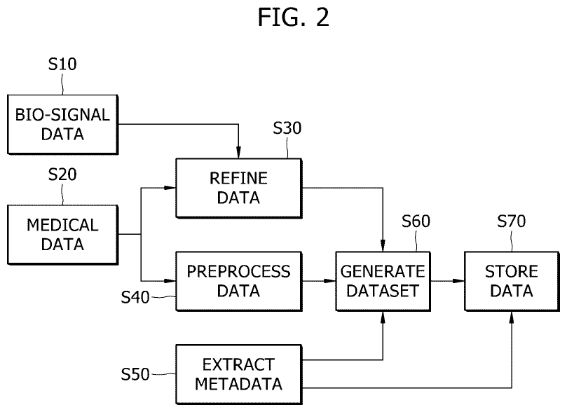

The processor 110 may store the dataset 125 in the memory 120 or database. FIG. 2 is a flowchart illustrating a data flow of the apparatus for refining and preprocessing bio-signal data according to an embodiment of the present invention. Referring to FIG. 2, the processor 110 may receive the bio-signal data 122 through the input unit 170 or the communication unit 130 (S10). The bio-signal data 122 may be received from the wearable device, the sensor, the medical device, etc.

In addition, the processor 110 may receive the medical data 121 through the input unit 170 or the communication unit 130 (S20). The medical data 121 may be received from at least one of the medical device, the wearable device, the terminal, the server, and the medical institution.

The processor 110 converts the bio-signal data 122 into the segment and refines the bio-signal data in units of segments (S30).

The processor 110 evaluates the quality of the bio-signal data 122 in units of segments to determine whether the bio-signal data 122 is suitable for learning.

When evaluating the quality of the bio-signal data, the processor 110 may analyze the morphology (such as waveforms of pulse waves) of the bio-signal data in the time, frequency, and nonlinear domains, and determine whether the bio-signal data 122 is suitable for analysis and learning based on the extracted feature values through the analysis of the sizes, ratios, characteristics, etc., of the frequency components.

The processor 110 may convert data into data for learning purposes for suitable segments. In addition, the processor 110 may interpolate data of some of the unsuitable segments using an interpolation method. The processor 110 may process data in units of segments, and interpolate data based on the signal pattern or the overlapping signal form of the data when the data is repeated or overlaps. For example, the processor 110 may interpolate, when the signal is in the form of a sine wave having a certain frequency, the data using the signal in the form of the sine wave having the certain frequency.

When the data interpolation is impossible, the processor 110 may use the segment as a negative sample.

The processor 110 may perform the conversion, normalization, filtering, and the data analysis based on the medical data 121 for the refined bio-signal data 122 in the time and frequency domains to preprocess the data (S40).

In addition, the processor 110 may classify the refined bio-signal data 122 as a specific group based on the medical data 121 and preprocess the bio-signal data 122.

The processor may extract the metadata 123 based on data generated during the refining and preprocessing of the data (S50).

The processor 110 may store the information and the result data 124 extracted during the refining and preprocessing. The processor 110 may manage the bio-signal data 122 included in the dataset 125 based on the metadata 123.

The processor 110 may extract the locations and numbers of R-peaks, the locations and numbers of systolic points, diastolic points, notches, etc., a heart rate, a peak-to-peak interval, etc., extracted therefrom as the metadata 123. In addition, the processor 110 may extract and store mapping relationship information between feature values in the bio-signal data 122 and the extracted metadata 123 as the metadata 123.

The processor 110 generates the dataset based on the refined and preprocessed bio-signal data 122 (S60).

In generating the dataset, the processor 110 performs the sampling on the preprocessed bio-signal data and metadata 123, processes the data, and converts the sampled data into the data form that can be provided to the machine learning or an artificial neural network to generate the dataset 125.

The processor 110 stores the result data 124, the dataset 125, and the metadata 123 constructed through the refining and preprocessing in the memory 120 or the database (S70).

The processor 110 manages the stored data and uses the stored data for learning. The processor 110 may search and extract the specific dataset 125 from the database using the metadata 123 as an index.

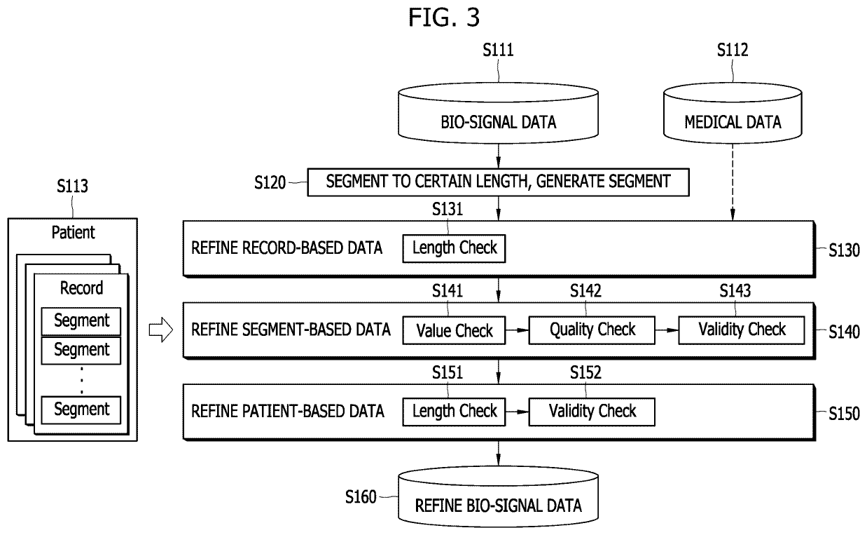

FIG. 3 is a flowchart illustrating a data refining process of the apparatus for refining and preprocessing bio-signal data according to an embodiment of the present invention.

Referring to FIG. 3, the processor 110 receives the bio-signal data 122 (S111) and receives the medical data 121 (S112).

The processor 110 divides the bio-signal data 122 into pieces of data with a certain length to generate the segments (S120).

In addition, the processor 110 may classify the medical data 121 as patient-specific data and generate segments with a certain length from each data (S113).

The processor 110 refines data based on the record of the bio-signal data (S130).

When refining the data, the processor 110 identifies the length of the segment (S131).

The processor 110 may refine data based on the segment (S140). The processor 110 identifies the data value of the segment (S141), identifies the quality of the segment (S142), and identifies the validity of the segment (S143).

In addition, the processor 110 may refine data based on the patient (S150). The processor 110 identifies the length of the segment based on the patient (S151) and identifies the validity (S152) to refine the data.

The processor 110 refines the bio-signal data 122 based on the record, the segment, and the patient, respectively, and stores the refined bio-signal data (result data) 124 in the memory 120 (S160).

In the process of refining the data in this way, the processor 110 may determine the quality as follows.

The processor 110 divides the data into pieces of data with a certain length to generate the segments, and when the data overlaps, is missed, or includes an outlier at a certain length (interval) and a certain frequency, may determine that the segment is not suitable for learning.

The processor 110 may interpolate, when data is repeated or the signal has a certain pattern, the data based on the repeated data or the certain pattern, even when the segment is not suitable. The processor 110 may perform an interpolation using a linear interpolation method, a spline interpolation method, etc., based on specific values of an interval within a segment.

In addition, the processor may interpolate a segment using at least one of an average value, a maximum value, a minimum value, and a median value using neighboring values in the interval within the segment.

For example, in the case of a bio-signal generated by a heartbeat, the processor 110 may identify whether there is a periodic pattern in the bio-signal by using the characteristics of having quasi-periodicity and using autocorrelation analysis according to time delay.

The processor 110 may generate feature vectors through dimension reduction of the bio-signal, such as PCA and t-SNE. The processor 110 may represent spatial features of the feature vectors by patterning the feature vectors through clustering.

Meanwhile, when the processor 110 may not find the pattern through the clustering, the processor 110 may determine that the bio-signal is contaminated by irregular noise (motion artifact, electromagnetic interference, ambient light interference, etc.).

The processor 110 may classify the quality of the data into binary or multiple by considering the quasi-periodicity, morphological characteristics, physiological characteristics, etc., of the bio-signal data.

For example, the processor 110 may classify the quality of data by using the range of physiologically possible heart rate, the range of physiologically possible of the highest and lowest blood pressure, the range of physiologically possible of blood pressure fluctuations within a certain interval, etc.

In addition, the processor 110 may determine the quality of the bio-signal data 122 by using the medical data 121.

For example, in the case of a subject with a specific disease such as an arrhythmia, since the bio-signal data of the subject may have different characteristics from those of a general patient, the processor 110 may use the bio-signal data to assign different quality grades to the bio-signal depending on the purpose of data collection and utilization. In the case of data that may affect the bio-signal data 122, such as surgery, treatment, and drug administration, the processor 110 may process the data separately by considering the data during the refining of the data, and may be used for the refining of the data in conjunction with the medical data 121.

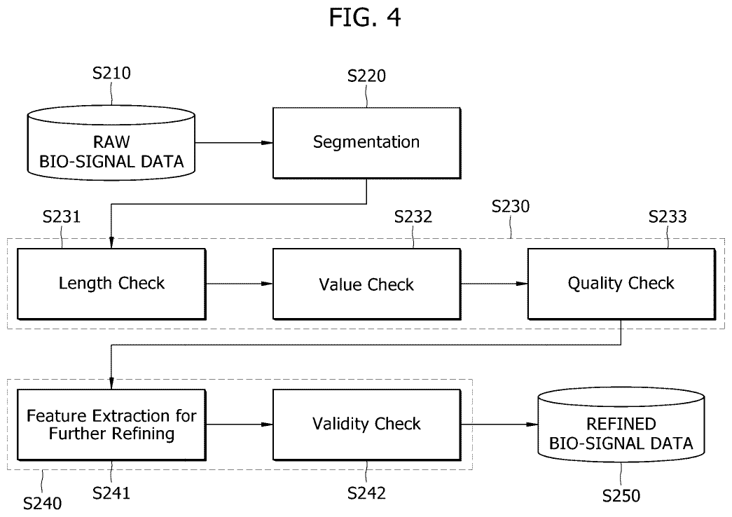

FIG. 4 is a flowchart illustrating a process of refining and preprocessing data of the apparatus for refining and preprocessing bio-signal data according to an embodiment of the present invention.

Referring to FIG. 4, the processor 110 acquires the bio-signal data 122 (S210) and generate segments with a certain length (S220).

The processor 110 refines data in units of segments (S230).

As described above, the processor 110 may identify the length (S231), identify the data value of the segment (S232), and identify the quality (S233).

The processor 110 may identify the length in the case of the record-based refining or the patient-based refining. In the process of identifying the quality, the processor 110 may identify the data value by analyzing whether the same value is repeated at a certain interval, etc.

In addition, the processor 110 may preprocess the refined data (S240). The processor 110 extracts features (S241) and identifies validity (S242).

The processor 110 may determine that bio-signal data 122 is invalid when the bio-signal data 122 is outside the measurable range or when the bio-signal data is a data value in the immeasurable range.

The processor 110 stores the refined and preprocessed bio-signal data (result data) 124 (S250).

The processor 110 performs the preprocessing such as the conversion, normalization, filtering, and the data analysis based on the medical data for the refined bio-signal data in the time and frequency domains.

The processor 110 performs the frequency conversion to standardize the sampling rate of the bio-signal data 122 and the normalization to constantly adjust the signal size in the preprocessing process. In addition, the processor 110 may acquire the features of the time, frequency, and nonlinear domains of the signal for the bio-signal data 122.

The processor 110 may first classify the bio-signal data 122 as a specific group based on the medical data 121 and process the refining and preprocessing differently for each group.

The processor 110 may detect features of an R-peak of ECG and a systolic point, a diastolic point, and a notch of PPG in the preprocessing process.

The processor 110 may feedback the heart rate and peak-to-peak interval extracted from the features to the refining process and use the feedback heart rate and peak-to-peak interval. The processor 110 may use the heart rate extracted from the bio-signal data to determine the quality of the corresponding data depending on whether the heart rate falls within the physiologically possible range.

The processor 110 may extract the metadata 123 based on the intermediate data and the result data generated during performing the refining and preprocessing in this way.

The processor 110 may extract the location and number of R-peaks, the location and number of systolic points, diastolic points, notches, etc., the heart rate, the peak-to-peak interval, etc., extracted therefrom as the metadata 123. In addition, the processor 110 may extract and store the mapping relationship information between the feature values in the bio-signal data and the extracted metadata 123 as the metadata 123.

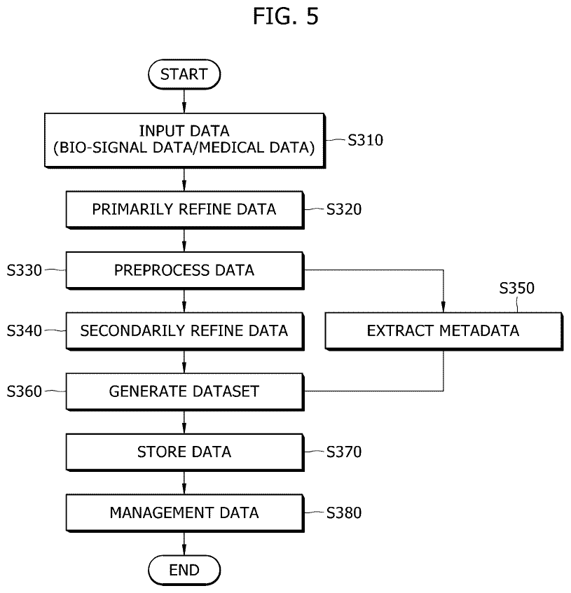

FIG. 5 is a flowchart illustrating a method of refining and preprocessing bio-signal data according to an embodiment of the present invention.

Referring to FIG. 5, the processor 110 acquires the bio-signal data 122 and the medical data 121 through the input unit 170 or the communication unit 130 (S310).

The processor 110 divides the bio-signal data 122 into pieces of data with a certain length to generate the segments.

The processor 110 refines the bio-signal data 122 (primary refining) (S320).

The processor 110 may refine data based on the record, refine data based on the segment, and refine data based on the patient in evaluating the quality.

In addition, the processor 110 may identify the length of the bio-signal data 122, evaluate the quality, and identify the validity.

The processor 110 may analyze the morphology (waveforms of pulse waves) of the time, frequency, and nonlinear domains of the bio-signal data 122, and analyze the characteristics according to the sizes and ratios of the frequency components to determine the quality of the bio-signal data 122.

In addition, the processor 110 may interpolate data based on the interpolation method when values are missed or the same values are repeated at a certain length or frequency.

The processor 110 preprocesses the refined bio-signal data 122 (S330).

The processor 110 may perform the preprocessing on the refined bio-signal data 122 through the conversion, normalization, filtering, and data analysis in the time and frequency domains.

The processor 110 may convert the frequency to standardize the sampling rate and perform the normalization to constantly adjust the signal size. In addition, the processor 110 may extract the features of the time, frequency, and nonlinear domains of the signal for the bio-signal data 122.

The processor 110 may feedback some data to the refining process during the preprocessing of the bio-signal data 122 to perform secondary refining (S340).

The processor 110 extracts the metadata 123 from the intermediate data or the result data generated during the refining and preprocessing of the bio-signal data 122 (S350).

The processor 110 generates the dataset for the bio-signal data 122 and the medical data 121 (S360).

The processor 110 stores the dataset in the memory 120 (S370). In addition, the processor 110 may transmit the dataset to the database through the communication unit 130.

The processor 110 may search for or manage data from the database using the metadata as the index (S380).

According to the apparatus and method for refining and preprocessing bio-signal data according to an aspect of the present invention, by standardizing and automating the process of refining and preprocessing the bio-signal data with various characteristics depending on the device or person, it is possible to effectively process a large amount of data.

According to the apparatus and method for refining and preprocessing bio-signal data according to an aspect of the present invention, by standardizing and automating the process of refining and preprocessing the bio-signal data with various characteristics depending on a device or person, it is possible to effectively process a large amount of data.

According to the apparatus and method for refining and preprocessing bio-signal data according to an aspect of the present invention, by selecting the high-quality bio-signal data, it is possible to secure a large amount of bio-signal data in various fields and easily manage and utilize the data.

According to the apparatus and method for refining and preprocessing bio-signal data according to an aspect of the present invention, it is possible to improve the quality of bio-signal data, facilitate the learning based on the bio-signal data, and improve the performance of the analysis results.

Although the present invention has been described with reference to embodiments shown in the accompanying drawings, it is only exemplary. It will be understood by those skilled in the art that various modifications and other equivalent embodiments are possible from the present invention. Accordingly, a true technical scope of the present invention is to be determined by the spirit of the appended claims.

What is claimed is:

1. An apparatus for refining and preprocessing bio-signal data, comprising:
a communication unit that receives bio-signal data or medical data; and
a processor that generates segments with a certain length from the bio-signal data, refines the data by determining a data value, quality, and validity of each of the segments, converts and normalizes the refined data to pre-process the data, and generates a dataset for learning or evaluation based on the refined and preprocessed data.

2. The apparatus of claim 1, wherein the processor classifies the bio-signal data based on at least one of units of records, units of the segments, and units of patients of the medical data and refines the data.

3. The apparatus of claim 2, wherein the processor identifies a length of the data in the units of the records to determine suitability of use of the data for training data.

4. The apparatus of claim 2, wherein the processor identifies a length of the data in the units of the patients, and determines validity to determine suitability of use of the data for training data.

5. The apparatus of claim 1, wherein the processor identifies cases in which the data overlaps, is missed, and includes an outlier at a certain length or a certain frequency of the segment to determine the quality depending on use of training data.

6. The apparatus of claim 5, wherein, when the data of the segment is repeated or the signal has a certain pattern, the processor interpolates the segment using specific values of an interval within the segment through an interpolation method.

7. The apparatus of claim 5, wherein the processor interpolates the segment using at least one of an average value, a maximum value, a minimum value, and a median value based on neighboring values in an interval within the segment.

8. The apparatus of claim 1, wherein the processor preprocesses the bio-signal data by performing frequency conversion to standardize a sampling rate of the bio-signal data and normalization to constantly adjust a signal size.

9. The apparatus of claim 1, wherein the processor acquires features of time, frequency, and nonlinear domains of the signal for the bio-signal data during a preprocessing process.

10. The apparatus of claim 9, wherein the processor feeds the features back to a process of refining data to perform secondary refining.

11. The apparatus of claim 1, wherein the processor extracts metadata from data generated during a refining and preprocessing process of the bio-signal data.

12. The apparatus of claim 11, wherein the processor samples refined and preprocessed bio-signal data to process the data, converts the processed data into training data to generate a dataset, and manages the dataset using the metadata as an index.

13. A method of refining and preprocessing bio-signal data, comprising:
acquiring, by a communication unit, bio-signal data or medical data;
generating, by a processor, segments with a certain length from the bio-signal data;
determining, by the processor, a data value, quality, and validity of each of the segments to refining the data;
converting and normalizing, by the processor, the refined data to preprocess the data; and
generating and storing, by the processor, a dataset for learning or evaluation based on the refined and preprocessed data.

14. The method of claim 13, wherein the refining of the data includes
refining the bio-signal data in units of records, and
the processor identifies a length of the data in the units of the records to determine suitability of use of the data for training data.

15. The method of claim 13, wherein the refining of the data includes refining the bio-signal data in units of the segments, and
the refining of the bio-signal in the units of the segments includes identifying cases in which the data overlaps, is missed, and includes an outlier at a certain length or a certain frequency of the segment to determine the quality depending on the use of training data.

16. The method of claim 15, wherein, when the data of the segment is repeated or the signal has a certain pattern, the refining of the bio-signal in the units of the segments includes interpolating the segment using specific values of an interval within the segment through an interpolation method.

17. The method of claim 15, wherein the refining of the bio-signal in the units of the segments further includes interpolating the segment using at least one of an average value, a maximum value, a minimum value, and a median value based on neighboring values in the interval within the segment.

18. The method of claim 13, wherein the refining of the data includes refining the bio-signal data in the units of the patients of the medical data, and
the processor identifies a length of the data in the units of the patients, and determines validity to determine suitability of use of the data for training data.

19. The method of claim 13, wherein, in the preprocessing of the data, the processor preprocesses the bio-signal data by performing frequency conversion to standardize a sampling rate of the bio-signal data and normalization to constantly adjust a signal size.

20. The method of claim 13, wherein the preprocessing of the data includes:
acquiring, by processor, features of time, frequency, and nonlinear domains of the signal for the bio-signal data; and
feeding the features back to a process of refining data to perform secondary refining.
